(19) United States
(12) Patent Application Publication
(10) Pub. No.: US 2009/0306455 A1
(43) Pub. Date: Dec. 10, 2009

(54) Method of Providing Magnetised Particles at a Location

(76) Inventors: Robert Andrew Slade, Oxon (GB); John Simon Owen Evans, Durham (GB); David Parker, Durham (GB); Russell Paul Cowburn, Buckinghamshire (GB); Michael Eaton, Berkshire (GB)

Correspondence Address: BLANK ROME LLP, WATERGATE, 600 NEW HAMPSHIRE AVENUE, N.W., WASHINGTON, DC, 20037, US

(21) Appl. No.: 11/922,021

(22) PCT Filed: Jun. 14, 2006
(86) PCT No.: PCT/GB2006/002189

§ 371 Date: Mar. 17, 2008

(30) Foreign Application Priority Data
Jun. 17, 2005 (GB) 0512402.9

(51) Int. Cl. A61N 2/10; H01F 1/00; H01F 1/04

(52) U.S. Cl. 600/12; 252/62.51 R; 252/62.55

(57) ABSTRACT

A method of providing magnetised particles at a location using particles which can be switched between magnetic and non-magnetic states by exposure to a suitable magnetic field. The method comprises conveying the particles in their non-magnetic state to the location; and then exposing the particles to the suitable magnetic field so that they switch to their magnetic state.

The invention relates to a method of providing magnetised particles at a location, such as in a body, both for medical and non-medical applications.

In recent years, there have been many advances in the treatment of disorders associated with aberrant cellular or tissue structures, by the selective targeting of the structures with appropriate therapeutic agents. In particular, in the context of tumour therapy, there have been significant advances in technologies that permit the localisation of appropriate chemotherapies at the tumour site. However, chemotherapy is often compromised by adverse systemic toxicity, which limits the dose of drug that can be administered, or is limited by the appearance of multi-drug resistance. One particular difficulty results from the need to control the cytotoxicity until after localisation at the tumour site, to prevent non-specific cell damage (Eaton, Immunoconjugates: Current Status and Future Potential, Journal of Drug Targeting, 2002; 10(7):525-527).

U.S. Pat. No. 6,514,481 describes the targeting of spherical magnetic nanoparticles less than 100 nm in diameter, to a cellular location, with subsequent application of a DC magnetic field, to destroy the targeted cells. The nanoparticles are prepared from iron oxide, e.g. Fe₂O₃, and an applied magnetic field of 7 Tesla is shown to be required to achieve in vitro cell death.

While the results achieved using this process are of interest, the requirement for a very strong magnetic field limits the suitability of the process for clinical applications due to the high cost of whole-body hardware for generating fields above about 2 Tesla, and the danger of damaging healthy tissue due to causing motion in non-localised naturally occurring or contaminant particles.

WO-A-01/17611 discloses the use of nanoparticles in a hyperthermia process that also requires the induction of shearing forces. The shearing forces are induced by applying an alternating magnetic gradient field. The only magnetic particles disclosed are metal oxides. The magnetic gradient field causes the particles to experience a translation force acting to move them along the field gradient; alternating the gradient field moves the particles in opposite directions, thereby inducing a “vibration” effect.

Halbreich et al., Journal of Magnetism and Magnetic Materials, 2002; 248: 276-285 describes a process referred to as “magnetocytolysis”. The particles used in the process are stated to be made of magnetite (iron oxide) and an optimum field oscillation frequency is indicated to be 1000 KHz. The mechanism of magnetocytolysis is not specified but is indicated to be related to locally generated intense gradient fields at the boundaries between regions with differing concentrations of magnetic nanoparticles, such as cell membranes.

U.S. Pat. No. 6,231,496 discloses the use of nanoparticles having a sharp end, which are to be embedded within the lining of the uterus by the application of a magnetic field. Microwave radiation is then applied to generate tissue heating resulting in the destruction of the uterine lining, to achieve sterilization.

U.S. Pat. No. 5,067,952 discloses the use of ferromagnetic particles for use in treating tumours by hyperthermia. The nanoparticles may also be caused to vibrate by the use of ultrasonic oscillation. Hyperthermia is carried out by applying an electromagnetic field at a frequency of 13.65 MHz. The destruction of the tumour is therefore a heat-based mechanism.

U.S. Pat. No. 5,236,410 discloses the use of hexaferrite particles in the treatment of tumours, via hyperthermia. The particles are said to be from 500 nm to 7 μm in size. The magnetic field frequency required to induce hyperthermia is approximately 500 MHz.

Although the above references show that magnetic particles have found use in therapeutic applications, there is still a requirement for improved processes for inducing cell death using magnetic particles, which do not rely on high field strengths and/or high frequency variation of the magnetic field, nor on procedures which induce localised or general temperature rise in tissue.

In WO-A-2005/011810 we describe an improved method for disrupting a material, the method comprising the steps of:
(i) localising one or more magnetic particles at or within the material; and
(ii) applying a magnetic field to the or each magnetic particle, to induce particle rotation and thereby disrupt the material, wherein the or each magnetic particle has intrinsic magnetization, said magnetization being stabilised by inherent magneto-crystalline anisotropy and/or by shape anisotropy and wherein the applied magnetic field direction or amplitude with respect to the material is varied over time.

One of the important aspects of this idea is the step of localising the magnetic particles at or within the material. It is advantageous to use ferromagnetic (hard) particles since weaker magnetic field strengths are required to cause cell damage but there is a potential problem which is that the use of such particles may cause unwanted agglomeration caused by the particles attracting each other due to their permanent magnetic dipole moment.

In accordance with the present invention, we provide a method of providing magnetised particles at a location using particles which can be switched between magnetic and non-magnetic states by exposure to a suitable magnetic field, the method comprising

conveying the particles in their non-magnetic state to the location; and

then exposing the particles to the suitable magnetic field so that they switch to their magnetic state.

In this context, by “non-magnetic states”, we mean states that possess zero net magnetisation when averaged across the volume of the particle.

We have realised that it is possible to take advantage of the inherent, magnetic properties of certain small particles which exhibit the property of being able to be switched between magnetic and non-magnetic states. Thus, by starting with the particles in their non-magnetic states they can be conveyed to the desired location without risk of agglomeration and at that point they can then be switched to their magnetic states.

Such particles were first described in “Single-Domain Circular Nanomagnets” Cowburn et al, Physical Review Letters, Vol. 83, No. 5, pages 1042-1045. A further discussion of these particles can be found in “Magnets and Nanometers: Mutual Attraction”, Koltsov et al, Physics World, July 2004, pages 31-35.

Typically, the particles comprise nanoparticles which means their largest lateral dimension is less than 1 micron. They are also typically of anisotropic shape although spherical particles could be used. Preferred shapes and sizes are:
(a) particles with a thickness (t) smaller than the lateral dimensions (x and y), but more than 5 nm. The lateral dimensions to be in the range 50-200 nm. They can be circular (x=y) or non-circular (x≠y).
(b) Spherical particles of diameters 50-200 nm (t=x=y).
(c) Particles where the thickness is similar to the lateral dimensions, but non-spherical, the largest dimension in the range 50-200 nm (t=x, y, x≠y). This includes spheroidal particles, that is particles that are sphere-like but not perfectly spherical.

These shapes and dimensions are chosen to ensure that the particles exhibit the required magnetic and non-magnetic states as will be explained in more detail below. At the same time, the particle sizes are chosen so that they can be easily conveyed to the required location.

The strength of the magnetic field used to cause a switch in the magnetic state of the particles will depend on their shape and material, but is typically 80 kA/m (a magnetic flux density of 0.1 Tesla).

Exposure to the magnetic field could be achieved simply by moving the body into a magnetic field or alternatively by generating a pulsed external magnetic field.

The invention is particularly applicable to the mechanical disruption techniques described in WO-A-2005/011810, incorporated herein by reference, and in that case, the magnetic field used to induce motion of the particles could also be used to cause them to switch from their non-magnetic to their magnetic states.

In some cases, it may be desirable to switch the particles back to their non-magnetic state. In that case, the method may further comprise exposing the particles in their magnetic state to a sequence of progressively weaker magnetic fields to cause particles to return to their non-magnetic state. Some particles (usually those that are thinner than they are wide) will require a decaying field that switches polarity to demagnetise it. Particles that are spherical, or have a thickness that is close in size to the diameter, will seif-demagnetise as soon as the field is reduced to zero.

A variety of materials may be used to fabricate the particles including Supermalloy (Ni78Fe18Mo4), Permalloy (Ni80Fe20) or Nickel.

Although the invention is particularly suitable for use in the human or animal body for treatment of disorders associated with the accumulation of a biological material or an aberrant cellular or tissue structure, the invention is also applicable more widely to use with methods for disrupting materials such as rust and other growths on inanimate bodies such as pipes and the like.

The method could be carried out in vitro as well as in vivo.

An example of a method according to the invention will now be described with reference to the accompanying drawings, in which:—

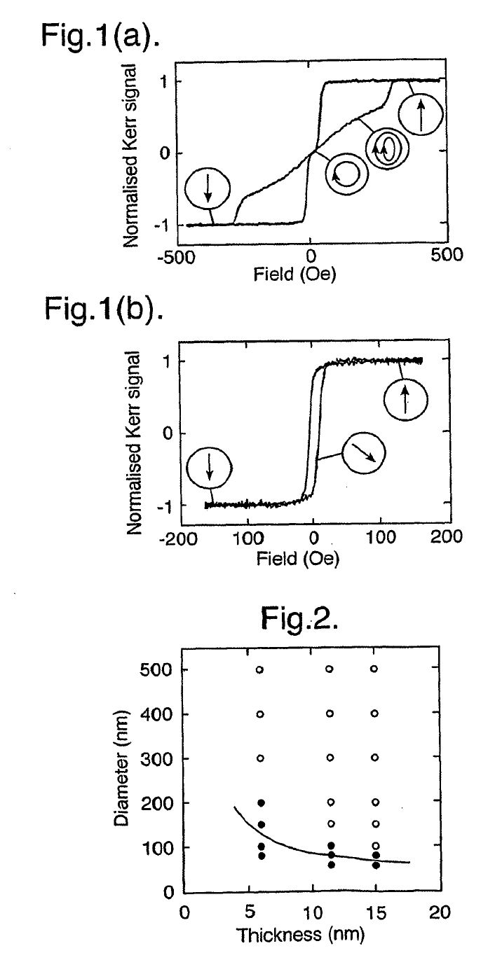

FIG. 1 illustrates hysteresis loops measured from nanomagnets of diameter (d) and thickness (t): (a) d=300 nm, t=10 nm; (b) d=100 nm, t=10 nm. (The schematic annotation shows the magnetization within a circular nanomagnet, assuming a field oriented up the page).

FIG. 2 shows an experimentally determined phase diagram with an open circle indicating a vortex and a black disk a single domain, the solid line showing a lower bound to the theoretical phase boundary between the vortex state (above the boundary) and a single-domain state (below the boundary);

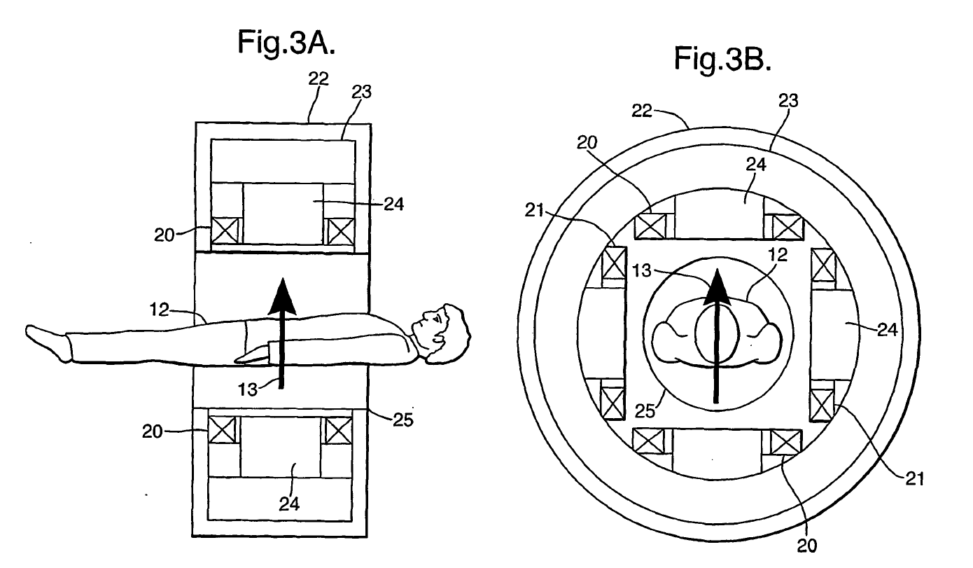

FIGS. 3A and 3B are a schematic longitudinal section and plan respectively of apparatus for carrying out a method; and,

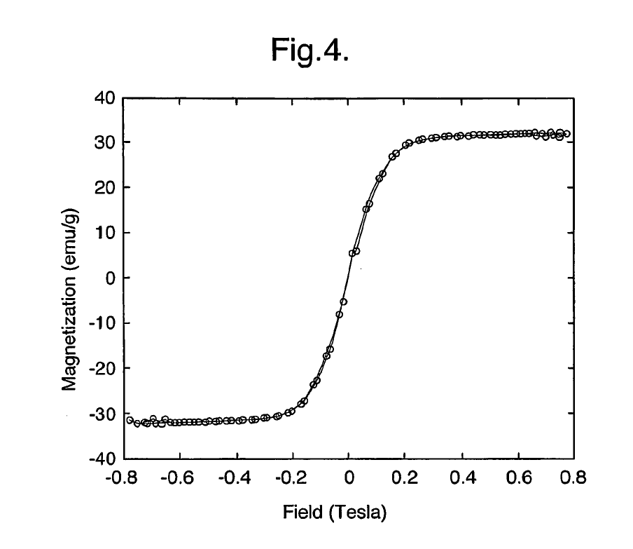

FIG. 4 illustrates the hysteresis loop for 80 nm Ni spheres.

The Cowburn et al paper mentioned above includes a detailed theoretical and practical analysis of the magnetic properties of nanomagnets made from supermalloy. In particular, and as can be seen in FIGS. 1a and 1b, the paper identifies that these magnets can have one of two classes of hysteresis loop which are shown in more detail in FIG. 1. The first (FIG. 1a) is the hysteresis loop for a nanomagnet with a diameter of 300 nm and a thickness of 10 nm and it can be seen that as the applied field is reduced from minus saturation, the nanomagnet retains full magnetic moment, until a critical field slightly below zero at which point nearly all magnetisation is lost. The nanomagnet is then in its non-magnetic state. The magnetisation then progressively reappears as a field is increased from zero, until positive saturation is achieved. The sudden loss of magnetization close to zero field is very characteristic of the formation of a flux closing micromagnetic configuration; the simplest of these is a vortex in which the magnetization vector remains parallel to the nearest edge at all points in the circular nanomagnet. Other configurations include a two domain state and a multi-domain state.

FIG. 1b illustrates the hysteresis loop for smaller nanomagnetics from which it can be seen that they do not exhibit the switching condition.

From these results, it is possible to generate a phase diagram (FIG. 2) which indicates whether or not a nanomagnet of a particular size can exhibit the vortex or non-magnetic state.

The size of the nanomagnet also depends upon the application. When used with biological material sizes up to a maximum dimension of 200 nanometers are preferred.

The magnetic particles can be made in any conventional manner of which examples are described in the Koltsov et al paper. For example, the particles can be chemically synthesized, or produced by condensing molten metal. Lithography is another option.

Further, in the case of applications with biological material, it is desirable to provide the particles with a bio-compatible coating. Examples include polyethylene glycol, ethyleneglycol copolymers, dextrin, polymers and copolymers of hydroxyalkyl(meth)acrylamide, for instance, hydroxypropylmethacrylamide, and copyolymers of styrene and maleic anhydride. Additional compounds include polyglutaric acid, carbohydrates and naturally occurring proteins such as albumin. The bonding can be either covalent or non-covalent. The coating will usually be applied to the particles prior to use in the method, however, it is envisaged that particles may attain a coating on administration, e.g. a coating of serum albumin.

The particles may be localised at a target site using any convenient means, including the use of a targeting moiety. The targeting moiety may be any suitable molecule that permits selective targeting to the target site. Examples of suitable targeting moieties include antibodies and receptor ligands, e.g. hormones.

In certain embodiments, the particles of the invention can be formulated to ensure proper distribution in vivo. For example, the blood-brain barrier may prevent the particles crossing into the brain and it may be preferable to deliver the particles in liposomes. Thus, in one embodiment of the invention, the particles are formulated in liposomes; in a more preferred embodiment, the liposomes include a targeting moiety.

Liposomes may be stealth liposomes that are long-lived in vivo. For methods of manufacturing liposomes, see, e.g. U.S. Pat. No. 4,522,811; U.S. Pat. No. 5,374,548; and U.S. Pat. No. 5,399,331. With regard to vaccine liposomes, and in particular, the so-called virosomes (for a review see Felnerova et al., 2004, Curr. Opin. Biotech.; 15:518-529), these may be manufactured as described in Moser et al (2003, Expert Rev Vaccines; 2:189-196), Bungener et al (2002, Biosci. Rep.; 323-338), and in Mastrobattista et al (2002, J. Liposome res.; 12:57-65). The liposomes may comprise one or more moieties which are selectively transported into specific cells or organs, thus enhancing targeted drug delivery (see, e.g. Ranade, W. 1989, J. Clin. Pharmacol.; 29:685). Exemplary targeting moieties include folate or biotin (see, e.g. U.S. Pat. No. 5,416,016.); mannosides (Umezawa et al., 1988, Blochem. Biophys. Res. Commun.; 153:1038); antibodies (Bloeman, P G. et al., 1995, FEBS Lett.; 357:140; M. Owais et al., 1995, Antimicrob. Agents Chemother.; 39:180); surfactant protein A receptor (Briscoe et al., 1995, Am. J. Physiol.; 1233:134), different species of which may comprise the formulations of the inventions; p 120 (Schreier et al., 1994, J. Biol. Chem.; 269:9090); see also Keinanen, K. & Laukkanen, M L. 1994, FEBS Lett.; 346:123; Killion, J J. & Fidler, I J. 1994, Immunomethods 4:273.

Further details of targeting processes are described in WO-A-2005/011810. This indicates that antibodies are the preferred targeting moiety.

In the context of treating a patient, it will be apparent to the skilled person how the particles should be administered to the patient. The particles will usually be administered by injection into the bloodstream, although alternative routes of administration, for example transdermal or ballistic delivery, may be suitable. Suitable pharmaceutically acceptable diluents, excipients or buffers will also be apparent to the skilled person.

In use, the particles are prepared in their non-magnetic state and optionally provided with a bio-compatible coating and then injected into the body where they are conveyed to the desired location. Again, this process is described in more detail in WO-A-2005/011810. Once at the desired location, the particles must be switched on so as to take up their magnetic state. This could be done by moving the body physically into the region of a magnetic field or alternatively switching on a previously inactive magnetic field. This latter approach is particularly convenient where a mechanical disruption process is to be carried out since the same magnetic field used for that can also be used to switch on the magnetic state of the particles.

An example of a suitable magnetic field generating apparatus is shown in FIGS. 3A and 3B. FIGS. 3A and 3B illustrate a set of coils 20,21 located on pole pieces 24 connected by a soft iron yoke 23 within a housing 22 having a bore 25 (22 and 25 may define a cryostat). The electromagnetics may be resistive or made from high (or low) temperature superconductor. In the case of resistive coils, water cooling will be needed to remove DC losses. In the case of superconducting coils, cryogenic cooling is needed. Rotation of the field direction 13 is caused by driving current in the coils 20, 21 in quadrature. This is a two phase structure but three or more phases could be implemented by using more electromagnetics. In the case of superconducting coils, the cryostat must be designed with a high cooling power to absorb AC losses. For this reason, high temperature superconductors may be preferred, as the cost of providing cryogenic cooling power is inversely proportional to temperature. Magnetic flux densities up to about 0.5 T are possible with a slew rate in the range of 1-5 T/sec, equivalent to a maximum field oscillation frequency of approximately 2.5 Hz.

In order to cause the mechanical disruption effect, and hence damage to cells in the vicinity of the particles, the particles must be caused to move and in order to do so they must experience a changing force and hence a changing magnetic field. In the simplest embodiment it is possible to simply pulse the magnetic field on and off. When the field is on, particles will attempt to rotate to align with the field. In the absence of an applied field, the particles' orientations will be random, therefore those particles that are already aligned with the field by chance will not experience any torque. It is therefore also desirable to change the direction of the applied field, so that all particles have a good chance of experiencing maximum torque. A rotating field will cause the particles to rotate to follow the field direction, thus maximising chances for cell damage. It is also desirable, but not required, to modulate the field amplitude over time, either continuously or in a pulsed fashion.

In one embodiment, the field direction or amplitude is varied at a frequency up to 100 Hz, preferably up to 50 Hz and more preferably up to 10 Hz. The change in field direction can be accomplished either by moving (eg: rotating) the patient within a static magnetic field or varying the field applied to the patient. The latter can be achieved by modulating currents in the electromagnet coils 20;21. The magnetic field direction should be directed perpendicular to the rotation axis, ie: across the working gap. Various other hardware options are feasible to generate such a field, each suited to a different mode of operation and field strength. In another embodiment, a uniform field upon which a small oscillating field gradient is imposed is applied.

After the mechanical disruption process, the magnetic nanoparticles could be returned to their non-magnetic state by applying a sequence of progressively weaker magnetic fields. FIG. 4 illustrates a hysteresis loop of some 80 nm Ni spheres. It can be seen that application of 0.2 T or so is enough to create full magnetisation, while reducing the field back to zero leads to zero remanence, because of the formation of the vortex state.

1: A method of providing magnetized particles at a location using particles which can be switched between magnetic and non-magnetic states by exposure to a suitable magnetic field, the method comprising:
conveying the particles in their non-magnetic state to the location; and
exposing the particles to the suitable magnetic field so that they switch to their magnetic state.

2: A method according to claim 1, wherein the particles exhibit a flux closing micromagnetic configuration in their non-magnetic state.

3: A method according to claim 1, wherein the particles comprise nano particles.

4: A method according to claim 1, wherein the thickness of the particles is about 5 nm.

5: A method according to claim 1, wherein the diameter of the particles is in the range 50-200 nm.

6: A method according to claim 1, wherein the particles have anisotropic shapes.

7: A method according to claim 1, wherein the thickness of each particle is at least an order of magnitude less than the diameter of the particle.

8: A method according to any of the preceding claims claim 1, wherein the suitable magnetic field exposes the particles to a magnetic flux density in the range of 0.01-2 Tesla.

9: A method according to claim 1, wherein the step of exposing the particles to the suitable magnetic field comprises moving the location into the magnetic field.

10: A method according to claim 1, wherein the step of exposing the particles to the single magnetic field comprises generating a pulsed magnetic field.

11: A method according to claim 1, further comprising applying a magnetic field to the particles at the location so as to induce particle motion and thereby disrupt adjacent material at the location.

12: A method according to claim 11, wherein the application of the magnetic field to induce particle motion also implements the step of causing the particles to switch to their magnetic state.

13: A method according to claim 11, wherein the material is a biological material.

14: A method according to claim 11, wherein the material is a cellular or tissue structure.

15: A method according to claim 14, wherein the cellular material is a mammalian cell.

16: A method according to claim 14, wherein the cellular or tissue structure material is a tumor.

17: A method according to any of the preceding lams claim 1, wherein the location is in a body.

18: A method according to claim 17, wherein the body is a human or animal body.

19: A method according to claim 1, wherein the method is carried out in vitro.

20: A method according to claim 1, further comprising exposing the particles in their magnetic state to a sequence of progressively weaker magnetic fields to cause the particles to return to their non-magnetic state.

21: A method according to claim 1, wherein the particles are made of Supermalloy (Ni₇₈Fe₁₈Mo₄), Permalloy (Ni₈₀Fe₂₀) or Nickel.

22: A method of locally treating a selected biological material in vitro or in vivo, comprising the steps of:
providing anisotropic particles adapted to being switched between magnetic and non-magnetic states;
locating the particles in their nonmagnetic states to a position within the biological material;
providing a magnetic field adapted to exposing the particles to an effective magnetic flux, wherein the magnetic flux causes at least some of the particles to switch to their magnetic states and move;
causing a disruption of some of the biological material adjacent the moving and magnetized particles; and
returning the particles to their nonmagnetic state.

23: A method of treatment according to claim 22, wherein the location is in a living human or animal body.

24: A method of treatment according to claim 22, wherein the magnetic field comprises a pulsed magnetic field.

25: A method of treatment according to claim 22, wherein the effective magnetic flux is in the range of 0.1-0.2 Tesla.
